(19) United States
(12) Patent Application Publication
(10) Pub. No.: US 2017/0354525 A1
(43) Pub. Date: Dec. 14, 2017

(54) DEVICE FOR NEURO-ORTHOPEDIC REHABILITATION

(71) Applicant: Viktoria Gennadyevna RYBCHYNSKAYA, Kaliningrad (RU)

(72) Inventors: Viktoria Gennadyevna RYBCHINSKYA, Kaliningrad (RU); Oleg RYBCHINSKY, Kaliningrad (RU)

(21) Appl. No.: 15/102,068

(22) PCT Filed: May 5, 2014
(86) PCT No.: PCT/RU2014/000320

§ 371 Date: Sep. 14, 2016

(51) Int. Cl. A61F 5/01; A61H 9/00; A61H 1/00

(52) U.S. Cl. CPC A61F 5/012; A61H 1/008; A61H 9/005; A61H 2205/10; A61H 2205/08; A61H 2205/062

(57) ABSTRACT

The invention relates to the field of medicine, specifically to neurology, and more specifically to devices for therapeutic gymnastics. A device consists of a combination of a loading system and an unloading system. Covers/pockets for pneumatic chambers of the unloading system are positioned longitudinally, in pairs and continuously, along a front surface and a rear surface of a load-bearing membrane. Support elements of a knee brace, made of two front portions and of two rear portions, interconnected by means of straps, are positioned in five sub-levels, wherein the two front portions of the brace are connected at the centers thereof by means of a linking component which is provided with an additional cushioning layer and with a half-ring in the center thereof, said half-ring serving for securing and changing the trajectories of elastic tensile components. A foot brace is removable and contains one longitudinal strap under the foot and three transverse straps which are connected to the longitudinal strap.

### CROSS REFERENCE TO RELATED APPLICATIONS

The present application is a National stage application from the PCT application PCT/RU2014/000320 filed on May 5, 2014. The present application claims priority to Design application 29/562,294 filed on May 25, 2016.

### TECHNICAL FIELD

The invention relates to medicine and, more specifically, to neurology, in particular, to devices for treatment and medical gymnastics for children and adults with the diseases accompanied with disorders of the locomotor apparatus or central movement regulation system.

### BACKGROUND ART

Known in the art there are the devices for straightening the limbs and corpus of the patient, as well for posture correction, hips, knees and ankle-joints fixing feet fixing and external rotation, using a different complex of elastic rods, semi-rigid and rigid connections, fixed to the patient's body, known as treatment device “Gravistan” (RU2131232 C1) and treatment device “Adele” (RU2054907C1, WO2005082295 A1). Said objects have the number of advantages, however, have a complex multiple configuration. The process of dressing, fitting and bringing of said devices into operation is laborious. The protection of the spine and joints is not specified. In cases of impossibility of self-retaining the poseby the patient, said devices do not allow the vertical adjustment.

Also known the devices for neuro-orthopedic rehabilitation, which contains the pressure shell in the form of fabric coverall with covers for the tensioning device of elastic chambers, made as the system of elastic chambers connected with the pumping equipment and placed along the outline of said suit (RU 2201177C2). In accordance with (RU 2266082C1) there is the tensioning suit with the pressure shell in the form of fabric coverall with covers for the tensioning device of elastic chambers, longitudinally placed along the outline of said suit in the line of the outer surface of the arms, legs and corpus, combined with the base fabric coverall with strips. The pressure shell has the joining in side seam, center seam, leg seam and shoulder seam, and the forepart with the central zipper and the lacing, placed on the front and the center of back part of the pressure shell.

Another coverall (WO201152756A1) is known with two elastic tubulous chambers: one of which is placed along the left flank and another along the right flank of the coverall. Moreover the coverall has the additional device for regulable chambers compression in the area, that meets both the position of the knee and/or elbow joints.

In the above described known devices, the main therapeutic effect is achieved through the creation of approximating (uniform) pressure on the patient's body, which leads to an improvement afferentation of the muscles and high proprioceptors irritation, and is an absolute advantage of these devices. At the same time, said effect significantly shortens the time of training in these devices and may serve in some cases contraindications to the use. As noted, in practice, these known devices do not allow loading or unloading of individual muscle groups and joints.

Insufficient pressure in the pneumatic chambers may have a weak supporting effect, while more pressure in pneumatic chamber can lead to complications as a result of peripheral circulatory disorders, to fluctuations in blood pressure and heart rate, which limits the functionality of use, requires a long time to get dressed, adjustment, removal.

### DISCLOSURE OF THE INVENTION

The present invention has for its principal object to overcome the problems described above, the expansion of the functionality of the device for neuro-orthopedic rehabilitation, while improving the convenience of its use, by reducing the time of dressing, adjustment, removal.

In accordance with the invention, the solution of the problem is achieved by the fact that the device comprising a pressure shell in the form of a fabric coverall with pockets-covers for elastic chambers (pneumatic chambers), longitudinally spaced along the contours of the suit, configured with possibility of connection to the pump device. In addition to the above this pressure shell comprises a front part with a central zipper, height and width regulator of coverall, shoulder, pelvic, knee, ankle and foot reference lines, interconnected with loading elements by means of supporting elements. The difference is, that the device is the combination of loading and unloading systems, in this case, the covers for pneumatic chambers of unloading system are placed longitudinally on the front and back surfaces of the pressure shell in pairs and continuously along the contours of said coverall on the level from the shoulder girdle to the ankle joints, namely: front: breast—front, inner hip—front, inner surface of the lower leg—front; back: center of the shoulder blade—back, externus hip—back, externus surface of the lower leg—back, in this case, pneumatic chambers are made with additional reinforcing layer and lengths, and fixing pneumatic chambers in the pocket covers are further enhanced with clasp “Velcro”. Moreover the supporting elements of the loading system of the shoulder girdle are placed in pairs on the chest in the third rib area, in pairs on the back of the near bottom corner contralateral shoulder blades, said support elements are connected with elastic rod passing through the semicircle in the protective peak scapular. In this case, the knee band supporting elements are formed of the two front parts and rear parts of two interconnected straps arranged in five sublevels, and two front parts of said band centered interconnected with strip, performed with an additional shock-absorbing layer and a semicircle in the middle, which serves to fix and change the trajectory of elastic rods. The foot band is removable, contains one longitudinal line beneath the foot and three transverse lines connected to the longitudinal line, and one transverse line located under the heel and ankle bandage is connected to the second transverse line located in the center of the arch, and the third transverse line is located on the bending fingers' line. While on said straps support elements are placed in pairs on the bending fingers' line in the middle of the foot fornix, in the center, rear of the calcaneus, and for fixing the ankle with a band provides additional adjustment elements, and correction of the position of the head was performed by paired pneumatic chambers, which pockets covers, located on the cap in the mastoid process area and in the collarbone area at the front of the suit.

The combination of loading and unloading systems, used in the claimed device makes the simultaneously verticalization possible due to external pneumoframework and dynamic motion correction, impeding the movement within the existing pathological movement patterns and facilitate the formation of physiological motor action.

Unloading system allows to provide assistance in the verticalization, create an adjustable support of Individual sections of the patient's body to protect from excessive load the spine and joints. Loading system ensures stable asymmetric selective loading of skeletal muscle and selective energy load certain types of movements.

The location of the pockets covers of pneumatic chambers of unloading system in the front and rear surfaces of the pressure shell longitudinally, in pairs and continuously along the contours of the overall from the shoulder girdle to the ankles area, namely, front: breast—front, inner hip—front, inner surface of the lower leg—front; back: center of the shoulder blade—back, externus hip—back, externus surface of the lower leg—back, eliminates critical bends of pneumatic chambers in which they lose their ability to hold the axial load, as well as allow a support joints in the most vulnerable areas: hip—and the rear side of the acetabulum, the knee joint—medially, without locking the possibility of joints movements.

Performing the pneumatic chambers with reinforcing layer can create pressure therein without changing the outer contour, which eliminates the pressure on the patient's body and creates an external pneumoframework. The removable performing of pneumatic chambers with the different lengths allows to fix them in any level of the pressure shell along the pockets covers, depending rehabilitation objectives and limitations of the patient.

The supporting elements of the loading system of the shoulder girdle, located on the chest in the area of the third rib, in pairs, on the back near the bottom corner of the blades contralateral and the supporting elements of the pelvic level, the support elements of knee band, located in five sublevels, as well as supporting elements of the ankle band and foot band can be connected by a system of elastic rods with adjustable tension force in the areas of its application, wherein said rods are made of different lengths and diameters, with sling or hook at the end. The adjustment of the tension force of the elastic rod is effected by changing the length of the support and fixing elements.

The claimed location of the supporting elements in the loading system allows for non-straight course of elastic rods, by connecting one elastic rod from two to five supporting elements that enhances the functionality of the device for physiological motor action arrangement. The supporting elements of the shoulder girdle allow to create an effective shoulders breeding, counteracting spasticity of pectoral muscles.

The performing of the lock for position of the head in the form of a cap with lacing in the occipital bone area with the tight fit for the size of the patient's head and two pockets covers for pneumatic chambers located in the mastoid area on the front of the suit and counter pockets covers located near the collarbone, can restrict flexion and extension in the cervical spine and head turns, adjusts the position of the head in cases of spasmodic torticollis, the instability of the cervical spine or muscular dystonia.

The performing the scapular from dense fabric, containing side peak with a semicircle, allows to protect the upper part of the shoulder joint from the pressure of elastic brace connected in pairs the supporting elements on the surface of the chest length and on the contralateral side of the rear part of the lower angle of the blade and at the same time to prevent offset of the thrust from a given trajectory.

The shoulder pad with the side peak is fixed to the shoulder joint by the clasp “Velcro” by adjustment the size of the coverall on growth, moreover said peak is made with an extra cushioning layer.

The performing of the knee bands comprising two front and two rear parts connected with the adjusting straps buckles allows to adjust the force of fixing the knee band, and perform the front band in the form of an upper and a lower crosspiece connected to fabric made with a cushioning layer and a semicircle in the middle, that enables to change the trajectory of fastening elastic rods.

The knee bands are fixed by clasp “Velcro”, located on two levels, that allows the adjustment to the growth of the patient, while there are additional holes for the passage of lines in the coverall interconnecting the front and rear parts of said bands inside the suit under the pockets covers of unloading system.

The fixation of the ankle is made as a band with a center buckle in the center for a tight fit. Along the perimeter of the ankle band there are the sewn support elements in the form of the buckles in pairs, one down and one up. Top buckles are used for fixing the elastic rods, the lower—for fixing the foot band. The fastening of the ankle band to the lower edge of trousers is performed by using the sewn strips into the trousers, moreover, there are two positions of fastening the ankle band to the edge of the trousers with adjustment on growth.

The foot strap is designed as a single system of removable bands, interconnected longitudinal with sling in the middle of the sole. Each strap of said band has separate adjustable locks that allow tightly attach them to the shoes.

The strap placed under the heel is made with three washers, two side and one back, these straps are used for fixation in the support elements the ankle band and allow the correction of varus or valgus position of the foot by changing the tension.

On the outer and inner side of the foot on the line of flexion of the fingers and in the center of the foot fornix, and in the center of the heel bone, behind, on the foot band there are the support elements of the loading system (adjusting buckles) for fixation to them of elastic rods. The support element located in the center of the heel bone, behind, allows using elastic rod to correct the position of the foot after the achilloplasty. Thus, the requested position of the foot in statics and dynamics is achieved by adjusting the heel band and load rods.

The pressure shell is adapted to fit Individual body size and fattiness of the patient by lacing located laterally along the entire length of coverall and in the back part of the coverall from neck to the waist, as well as by using of the straps located in the hip and ankles area.

Using the pressure shell in the form of fabric tightly laced coverall on the patient's body has the effect of soft corseting.

The adjustment of the size of the coverall on growth is effected by the buckle “Velcro” type located on the shoulders and on the trouser bell bottom.

Using pressure shell in the form of a fabric coverall saves time when dressing on and when removing the coverall, with all the elements of the loading and unloading systems after their pre-attenuation and deflating the air remains in place and re-use does not require installation, you just tighten the adjustment elements of the loading system and fill the air in the pneumatic chambers in the pockets covers of unloading system.

Further objects and advantages of the present invention will be understood from the following detailed description of a specific exemplary embodiment thereof and the accompanying drawings, wherein:

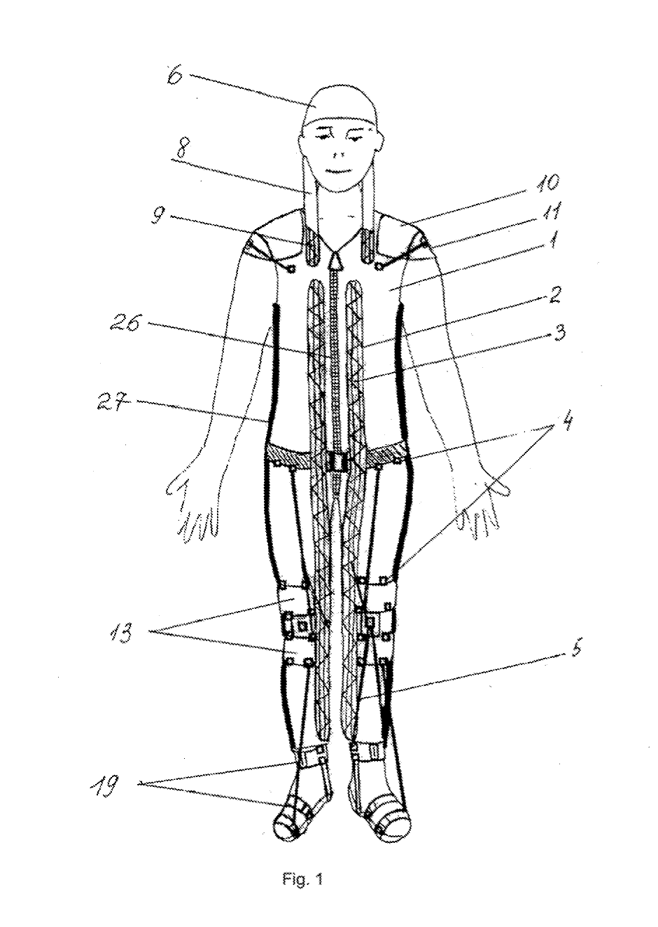

FIG. 1—is a general front view of the device for neuro-orthopedic rehabilitation

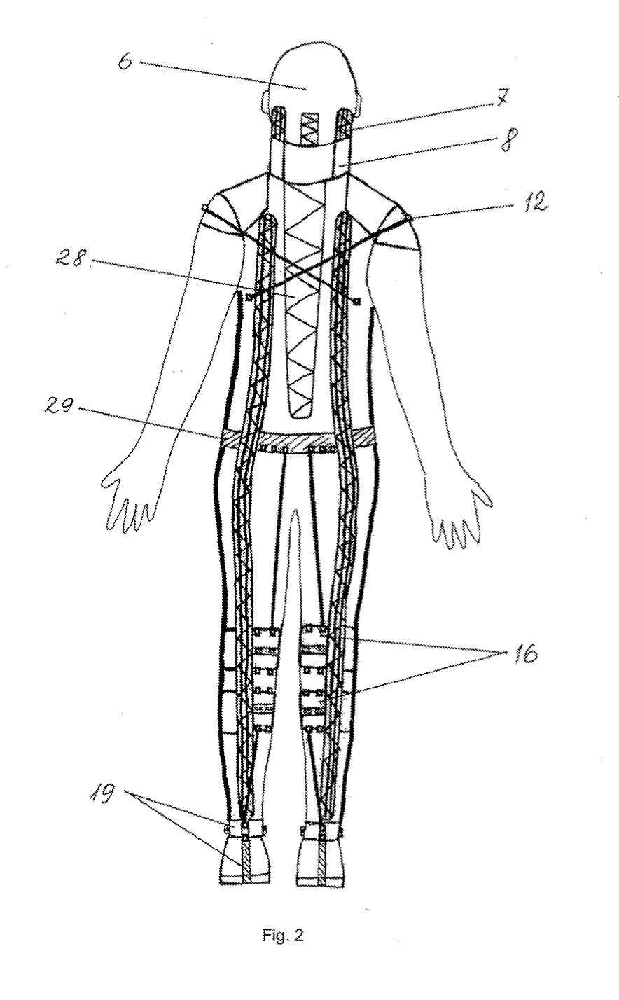

FIG. 2—is a general back view of the device for neuro-orthopedic rehabilitation

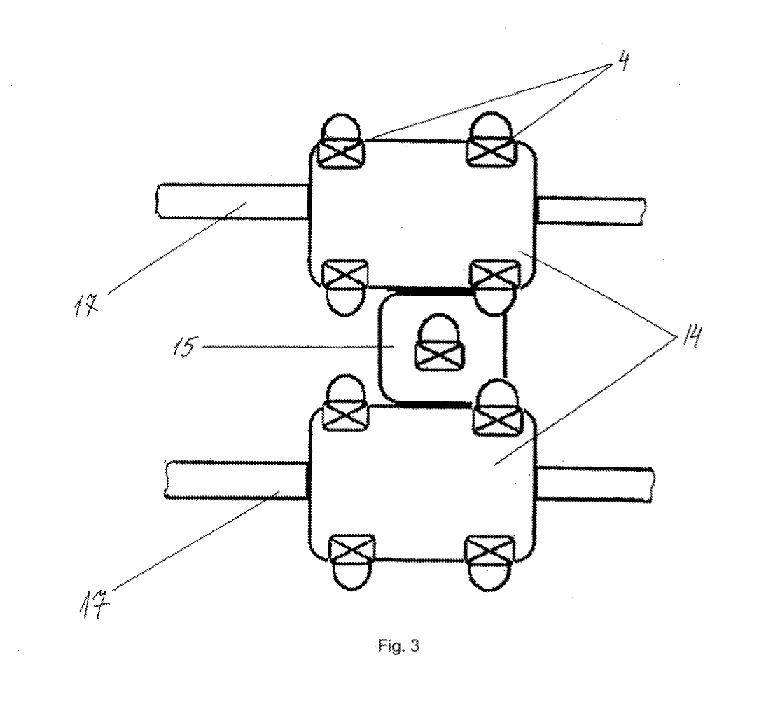

FIG. 3—is a knee band—front view

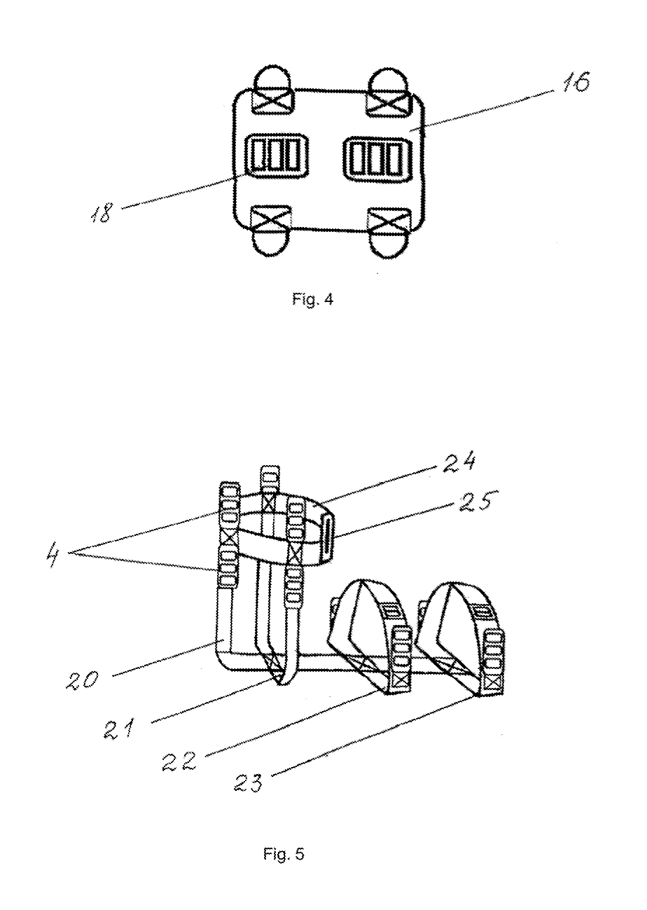

FIG. 4—is a knee band—back view

FIG. 5—is a removable foot band—side view.

### DETAILED DESCRIPTION, CONFIRMING THE INVENTION IS POSSIBLE

The device for neuro-orthopedic rehabilitation consists of the pressure shell 1 in the form of the fabric coverall (FIG. 1), four pockets covers 2 longitudinally placed along the contours of the coverall, front: breast—front, inner hip—front, inner surface of the lower leg—front; back: center of the shoulder blade—back, externus hip—back, externus surface of the lower leg—back, installed therein pneumatic chambers 3 (located under the lacing) made of an elastic material with an additional reinforcing layer, being connectable to pumping device (not indicated in the figures).

The loading system contains elastic rods 5 and supporting elements 4 for fixing them, located on the reference lines: shoulder girdle, pelvis, knee, ankle, foot level, while the supporting elements 4 may be in the form of adjustment buckles and semi-rings.

The lock of the head position is made in the form of the cap 6, contains two pockets covers for the pneumatic chambers 8 (FIG. 1,2), in the mastoid area-pockets covers 7 and mating pockets covers 9, located on the front part of the pressure shell 1 in the area of the collarbone.

The shoulder pad 10 is made with a peak 11, which contains an additional cushioning layer and the sewed semicircle 12.

The knee bands 13 contain two front parts 14 (FIG. 3) and two back parts 16 (FIG. 4), interconnected by the strap 17 with adjustment buckles 18, wherein the front parts 14 (upper and lower) are connected by the sewed in fabric strip 15 (FIG. 3), made with an extra cushioning layer.

The knee bands 13 have five sublevels of fixing the supporting elements 4, located on the front 14 and back 16 surfaces of the knee bands 13 (FIG. 4): two levels are at the upper part 14 of the knee band, one is in the center of the strip 15 and two levels are in the lower part 14.

The foot band 19 (FIG. 5) is removable, contains one longitudinal strap 20 located under the foot and three transverse straps connected to the longitudinal strap 20, while one transverse strap 21 is located under the heel and is connected with the ankle band, the second transverse strap 22 is located in the center of the foot, and the third transverse strap 23 is located on the fingers flexion line, while on said strips the support elements 4 are placed in pairs on the fingers flexion line, in the center of the foot, in the center of the calcaneus from backside.

The foot band 19 is fixed to the lower part of the suit on the edge of the pants by using the ankle band 24 with central buckle 25 located in front. Along the perimeter of the ankle band 24 there are the supporting elements 4 in the form of adjustment buckles: two pairs on the side surfaces and one pair on the back.

The device also has a central buckle 26 in the form of zipping (FIG. 1), side lacing 27 (FIG. 1 and FIG. 2), shown by the solid line on the sides and extra lacing 28 on the back to fit the pressure shell width. The strap 29 strengthens the pelvic sling line of supporting elements.

The device for neuro-orthopedic rehabilitation can be used as follows. In the first phase the features of motor disorders of the patient a reanalyzed, the objectives of rehabilitation are determined and the necessity for additional protection of the spine and joints is identified.

Select the appropriate size of the coverall, fully reveal the central clasp 26 and means for controlling the size on length and width, set the pneumatic chambers 3 in the pockets covers 2 of the pressure shell 1, then fix the elastic rods 5 to the supporting elements 4. Said elements are adjusted in combination, needed to achieve the objectives of rehabilitation.

Put on the coverall, fasten the front clasp 26, and then adjust the length and width of the pressure shell 1 by said means. Set the shoulder pads 10, knee bands 13 in the needed position according the growth of the patient, fix them using the adjustment buckles. Set the foot bands 19 and the ankle bands 24 are fixed with the central buckle 25. The adjustment of the unloading system of the pressure shell lis effected by creating the necessary pressure in the pneumatic chambers 3, and the adjustment of the loading system is carried out by the force of the tension of the elastic rods 5 using the adjustment buckles.

If necessary, fix and/or correct the position of the head by putting on the cap 6, insert the pneumatic chambers 8 in the pockets covers 7 in the mastoid area and in mating pockets covers 9 in the front part of the coverall in the collarbone area, tighten the laces of the pockets covers 7,9 and of the cap, create the necessary pressure in the pneumatic chambers 8 to determine the vertical position of the head. The device is ready for use.

In the course of the treatment to perform certain exercises the tension of the elastic rods 5 and the pressure in the pneumatic chambers 3 may be changed to facilitate, or to create the additional exertion for certain movements.

After finish the exercises the tension of the elastic rods 5 is eased off, the air from the pneumatic chambers 3,8 is going flat, the fixation in the regulating elements of the loading system and in the means of regulating the size on the length and on the width is also eased off. The coverall is put off from the patient, all the elements of the unloading and loading systems are left on the same place. By re-using you need only tighten the adjustment elements of the loading system and fill the air in the pneumatic chambers.

### Example 1

Male patient M., 9. Diagnosis: infantile cerebral paralysis, Little disease, advanced residual stage, medium severity, cerebellar deficiency syndrome, pseudobulbar dysarthria syndrome, planovalgus deformity. By verticalization rough flexion stand occurs in the hip and knee joints, there is no footing. Problem at the moment to keep balance while standing, formation the correct stereotype of walk.

Installing two rear pneumatic chambers along the entire length of the covers-pockets in the contours of the coverall on the level from the shoulder girdle to the ankles, from to the center of the blade—rear, outer hip—rear, the outer surface of the lower leg—rear, fixing with the lacing. For extension of the hip and knee joints four long rods are fastened to the supporting elements, an upper elastic rod is passed through the right semicircle strip of the knee band, thus encircle the knee joint from the outside and the inside, passing through the semicircle of the rear upper part of the knee band and securing the tips of the sling to the buckles on the sacrum to the right, while stretching the knee as well as hip joints. Wrap around the lower elastic rod from the semicircle strip of the knee band the knee joint on the outside and on the inside, passing through the semicircles of the rear part of the knee band, fixing to the upper ankle straps buckles on both sides. Repeat the steps on the left side.

In a prone position put on the suit on the patient, control the fullness and length according the main algorithm, fixed the shoulder pad, fix the knee band, fix the foot band on the shoes of the patient, tighten the heel strap of the foot band on the inside, creating a moderate varus position of the foot. Inflate the pneumatic chambers to two atmospheres, pull elastic rods, verticalize the patient. For the shoulders dilution pass the elastic rod through the semicircle of the scapular protective peak and pull.

The child is standing on straight legs by gravity, the movement in the joints are possible, let's begin the exercises.

25 sessions were held in the coverall with the following results: a significant reduction in spasticity in flexor and adductor muscles thigh, walking on his knees independent, self-standing, walking with support with one hand.

### Example 2

Female patient V., 5. Diagnosis: infantile cerebral paralysis, atonic-astatic form, late residual stage, mild, kyphoscoliosis of thoracic and lumbar spine, dysplasia of the left hip joint. The child walks on their own, the configuration of walk is broken, limping on his left leg, recurvation of the knee joints.

Install two rear pneumatic chambers: the right to the level of the sacrum, left—to mid-hip (unload the spine and left hip). To eliminate hyperextension in the knee joints four long rods are fixed to the supporting elements, while the upper elastic rod with the center in the popliteal fossa of the right leg is passed through the upper part of the semicircles of the front knee band inside and outside and is attached to the supporting elements of the pelvic reference line to the right in the front, making it easy to bend knee and to remove the hip.

The lower elastic rod of the popliteal fossa encircle the knee joint from both sides, it is passed through the semicircles in the lower front part of the knee band and is attached to the upper ankle strip buckles from both sides. Repeat all the steps on the left. Put on the coverall according the main algorithm. For this form of infantile cerebral paralysis the tension of the elastic rods performed with mild to moderate force.

14 forty minutes sessions were held in the coverall with the following result: improving the removal of the hip, matching the tone of the antagonist muscles, strengthening the muscles of the back, a significant decrease in limping.

Thus, said device for neuro-orthopedic rehabilitation through a combination of loading and unloading systems, correcting posture and motion path, can create individual rehabilitation facility that meets the specific objectives of rehabilitation and takes into account the nature of the disease and the possibility of the patient at a certain stage, that can significantly extend the application of said device in neuro-orthopedic rehabilitation and facilitate its use, excluding the possibility of complications and traumatism in the course of treatment, so that the unit is universal for all forms and degrees of motor disorders, providing the widest range of rehabilitative opportunities—from helping in verticalization to workout the exact trajectory of motion while improving the convenience of its use.

What is claimed is:

1-9. (canceled)

10. A device for neuro-orthopedic rehabilitation, comprising:
a combination of an unloading system and a loading system,
the unloading system comprising a supporting system for a user wearing the device for neuro-orthopedic rehabilitation, the unloading system providing support when engaged such that the user unable to otherwise stand is able to stand and perform rehabilitation exercises,
the loading system comprising a tensile system for the user wearing the device for neuro-orthopedic rehabilitation, the loading system providing muscle training for the user,
the unloading system being adjustable for strength and assistance and the loading system being adjustable for tensile force provided, as the user achieves progress in rehabilitation, and

11. The device of claim 10, wherein said unloading system is unengaged and said user is in a laying position and unloaded state.

12. The device of claim 10, further comprising:
a pressure shell, the pressure shell having a fabric coverall with a plurality of pocket covers, the plurality of pocket covers comprising at least one removable elastic chamber that acts as a pneumatic chamber, the plurality of pocket covers being longitudinally disposed along a plurality of contours of the fabric coverall,
the pressure shell having a front part with a central zipper,
a means for adjusting the fabric coverall,
the fabric coverall having a front surface and a back surface, and
the pressure shell having a plurality of supporting lines that interconnect with a plurality of loading elements via a plurality of supporting elements.

13. The device of claim 12, wherein a correction of a head position is performed by a pair of pneumatic chambers, said pneumatic chambers having pocket covers that are located on a cap in a mastoid process area and in an area of a user's collarbone on the front surface of the fabric coverall.

14. The device of claim 13, wherein the cap is formed with a lacing from the back surface of the pressure shell.

15. The device of claim 12, wherein the at least one removable elastic chamber that acts as a pneumatic chamber in the plurality of pocket covers is fixed by a hook and loop clasp and strengthened by a lacing of the plurality of pocket covers.

16. The device of claim 12, wherein on the front and back surfaces of a hip of the fabric coverall and on ankles of the fabric coverall the supporting elements are strengthened by a sling with an adjusting buckle.

17. The device of claim 12, further comprising additional holes for a plurality of sling passages in the fabric coverall for a knee band's fixation, interconnecting a front and a back part of said knee band on an inner side of the fabric coverall under the plurality of pocket covers of the unloading system.

18. The device of claim 12, wherein the at least one removable elastic chamber that acts as a pneumatic chamber is connected to a pump device.

19. The device of claim 12, wherein the at least one pneumatic chamber is made so that it becomes more rigid without changing its shape when air is pumped in it, thereby the shape remains comfortable while providing additional strength and support.

20. The device of claim 12, wherein the unloading system is assembled of elements as follows:
the plurality of pocket covers contain the at least one removable elastic chamber that acts as the pneumatic chamber and are located longitudinally along a front surface and a back surface of the pressure shell in pairs along with a plurality of contours in the fabric coverall from a girdle at the shoulder to the ankle,
the plurality of pocket covers run along the fabric coverall on the front from a front breast position to a front inner hip position to a front inner surface of lower leg position,
the plurality of pocket covers run along the fabric coverall on the back from a back center of a shoulder blade position to a back externus hip position to a back externus surface of lower leg,
the pneumatic chambers are made with an additional reinforcing layer and lengths,
fixing the pneumatic chambers in the plurality of pockets covers are further enhanced with a hook and loop clasp, and
each element of the unloading system is removable and is adjustable for strength and assistance or tensile force provided.

21. The device of claim 20, wherein the shoulder of the pressure shell has a shoulder pad and the shoulder pad has a peak that provides an additional cushion layer.

22. The device of claim 12, wherein the loading system is assembled of elements as follows:
the fabric coverall has a shoulder girdle, a chest area near a third rib, a bottom corner of blades contralateral on the back, a pelvic level, a knee band with five sublevels, an ankle band, and a removable foot band,
a plurality of supporting elements run between and connect the shoulder girdle, the chest area near the third rib, the bottom corner blades contralateral on the back, the pelvic level, the knee band with five sublevels, the ankle band, or the removable foot band,
wherein the loading system is a tensile system for the user wearing the device for neuro-orthopedic rehabilitation which provides muscle training for the user, and
each element of the loading system is removable and is adjustable for strength and assistance or tensile force provided, as the user achieves progress in rehabilitation the device for neuro-orthopedic rehabilitation can be adjusted for the user.

23. The device of claim 22, wherein the shoulder of the pressure shell has a shoulder pad and the shoulder pad has a peak that provides an additional cushion layer.

24. The device of claim 22, wherein:
the removable foot band, contains one longitudinal strap located under the foot and three transverse straps connected to the one longitudinal strap,
a first of the three transverse straps is located under a heel of the user and is connected with the ankle band,
a second of the three transverse straps is located in a center of a foot of the user,
a third of the three transverse straps is located on a fingers flexion line, and
the supporting elements are placed in pairs on the fingers flexion line, in the center of the foot, in a center of a calcaneus from a backside, and with additional adjustment elements for fixing with the ankle band.

25. The device of claim 22, wherein the supporting elements are connected by a system of elastic rods with adjustable tension force at the shoulder girdle, the chest area near the third rib, the bottom corner blades contralateral on the back, the pelvic level, the knee band with five sublevels, the ankle band, or the removable foot band.

26. The device of claim 25, wherein an end of the elastic rods may comprise a hook and sling or two slings.

27. The device of claim 25, wherein said rods are made of different lengths and diameters and said rods comprise a sling or a hook at one end.

28. The device of claim 25, wherein the supporting elements are in semicircular form on a back part of the knee band and provide additional fixation points of non-lineal stroke of the elastic rods.

29. The device of claim 25, wherein supporting elements are sewn in a form of buckles along a perimeter of the ankle band, said buckles being in pairs such that one buckle is a top buckle, and another buckle is a lower buckle, said top buckles are used for fixation of the elastic rods and said lower buckles are used for fixation of slings of the foot band.
